(19) United States
(12) Patent Application Publication
(10) Pub. No.: US 2015/0306413 A1
(43) Pub. Date: Oct. 29, 2015

(54) Magnetic Lumbar Orthosis with Rigid Support

(71) Applicant: Lev Tripolsky, Redwood City, CA (US)

(72) Inventor: Lev Tripolsky, Redwood City, CA (US)

(21) Appl. No.: 14/631,726

(22) Filed: Feb. 25, 2015

Related U.S. Application Data
(63) Continuation-in-part of application No. 13/542,595, filed on Jul. 5, 2012, now abandoned.
(60) Provisional application No. 61/505,110, filed on Jul. 6, 2011.

(51) Int. Cl. A61N 2/06; A61F 7/02; A61N 2/00; A61F 5/02

(52) U.S. Cl. CPC A61N 2/06; A61F 5/028; A61F 7/02; A61N 2/008; A61F 2007/0027

(57) ABSTRACT

A lumbar orthosis, comprising a rigid thermoplastic plate molded to the contours of a patient's back and at least one permanent magnet located on the orthosis that can provide magnetic therapy for patients with severe back problems, and further comprising adjustable supplementary fasteners that enable the patient to tighten or loosen the orthosis without removing it. In an embodiment, the lumbar orthosis also comprises a hot/cold pack to provide therapeutic heat or cold to the injured area.

### CROSS-REFERENCE TO RELATED APPLICATIONS

The present application is a continuation-in-part of application Ser. No. 13/542,595, filed Jul. 5, 2012, which claims the benefit of U.S. provisional patent application No. 61/505,110, filed Jul. 6, 2011, which are both incorporated herein by reference in their entirety.

### BACKGROUND

1. Field of Invention

The invention pertains to lumbar supports, specifically lumbar supports that use magnetic elements for therapeutic purposes.

2. Prior Art

Magnetic therapy is well known for its benefits in the treatment of musculoskeletal disorders. The application of magnets to a painful area is known to decrease pain and improve circulation. Since lower back pain is a significant problem for many people, there are several designs in the prior art for lumbar supports that incorporate magnets.

U.S. Pat. No. 4,480,596 to Shumiyashu discloses an elastic belt that fits around the lower back with magnets incorporated into it. The magnets are removably mounted to a flexible ABS sheet. The belt is relatively narrow, and thus, while it is useful in mild cases of back pain, it does not provide enough support for the back in cases of severe pain or instability.

U.S. Pat. No. 5,450,858 to Zablotsky et al. discloses an inflatable lumbar support that includes permanent magnets with a specified magnetic field pattern in order to improve circulation. The inflatable support provides a static stretch to the lower back muscles and improves back stability by providing compression. However, similarly to the Shumiyashu invention, the Zablotsky back support does not provide a sufficiently rigid orthosis for severe back pain or instability, which means that it can only provide magnetic therapy to patients with mild back pain.

### SUMMARY OF THE INVENTION

It is the primary object of this invention to provide a rigid lumbar orthosis that incorporates permanent magnets for magnetic therapy. This enables magnetic therapy to be used in cases of severe back pain or instability, which enables patients with more serious back problems to benefit from its effects.

It is also an object of this invention to provide a lumbar orthosis that incorporates a pocket into which either a rigid backplate or a hot/cold pack may be inserted, and magnets attached to the orthosis for magnetic therapy.

The present invention preferably comprises a belt that is at least 6″ wide with a fastener on each end. There is a pocket attached to the belt approximately at the halfway point along its length. A rigid backplate that conforms to the contours of a patient's back is inserted into the pocket. At least one permanent magnet is attached to either the pocket area of the belt or to the rigid backplate. The at least one permanent magnet preferably emits a magnetic field of at least 400 Gauss. In an embodiment, a gel hot/cold pack may be provided as well, and it may be possible to remove the rigid backplate from the pocket and to insert the gel hot/cold pack into the pocket for heat/cold therapy. The gel hot/cold pack is preferably approximately the same size as the pocket.

The permanent magnet or magnets are preferably made of the Y15 alloy, but may be made of any material that provides the required magnetic field.

In an embodiment, the magnetic lumbar orthosis comprises a plurality of permanent magnets arranged in an array. In the preferred embodiment, the array is a 5×5 rectangular array.

In an embodiment, the belt also comprises a front pocket located in the area opposite the pocket, and comprises a semi-rigid front plate inserted into the front pocket to provide additional support to the patient. The front plate is preferably made of LDPE. The front pocket or the front plate may also have at least one permanent magnet attached. In the preferred embodiment, the front pocket comprises six permanent magnets arranged in a 2×3 rectangular array.

The belt is preferably made of TheraHeat heat retention fabric to provide therapeutic heat to the patient's back. Any other heat retention, heat-reflection, or heat-generating fabric may be used as well.

In an embodiment, the belt comprises at least one supplementary fastener that may be used to tighten or loosen the belt without disturbing either the first or the second fastener. The supplementary fastener may comprise a short strap attached to the belt on one end. The strap comprises a hook-and-loop fastener on the free end, and a second patch of a hook-and-loop fastener is attached to the belt close to the free end, so that the strap may be used to tighten or loosen the belt.

In another embodiment, the supplementary fastener comprises two flaps attached at a short distance from each other. Each flap comprises at least one eyelet hole through which a lace may be threaded. A lace may then be threaded through both flaps and used to tighten or loosen the belt as needed without disturbing the main fastener.

The description herein provides preferred embodiments of the present invention, but should not be construed as limiting its scope. Variations in the type of rigid lumbar orthosis utilized, the type, shape, and quantity of the magnets used, the strength of the magnetic field, and the type of fasteners used for the orthosis, can all be incorporated into the present invention. The scope of the present invention should be determined by the appended claims and their legal equivalents, rather than the examples given.

### DESCRIPTION OF THE DRAWINGS

FIG. 1 is a view of the lumbar orthosis showing the pocket and the rigid backplate.

FIG. 2 is a semi-transparent view of the lumbar orthosis laid flat.

FIG. 3 is a view of the lumbar orthosis being worn by a patient, showing the magnets.

FIG. 4 is a view of an alternate embodiment of the lumbar orthosis.

### DETAILED DESCRIPTION OF THE PREFERRED EMBODIMENT

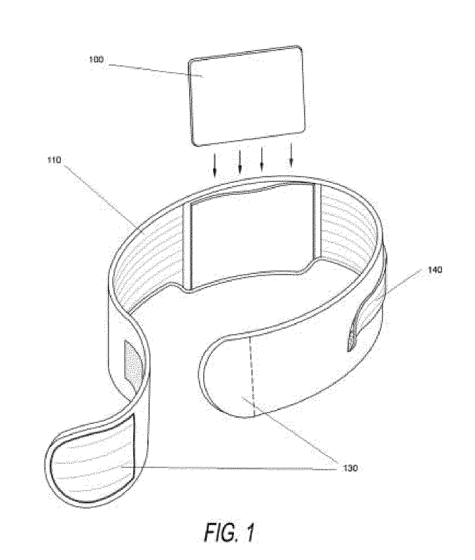

FIG. 1 shows a view of the preferred embodiment of the lumbar orthosis. Rigid backplate 100 is inserted into a pocket on belt 110. The rigid backplate 100 is preferably made of a thermoplastic material typically used for orthoses, as is well known in the art of orthotics. The material can be heated and molded to the patient's individual contours for greater comfort and better support. The thickness of the rigid backplate 100 is preferably 1.5 mm to prevent excessive flexing. However, the rigid backplate may be any other thickness or strength as required by the patient.

In the preferred embodiment, the rigid backplate 100 should be large enough to prevent any flexing of the patient's back. This applies to both the width and the length of the rigid backplate. In the preferred embodiment, the length of the rigid backplate should be enough to support the patient's entire lumbar spine; generally, a length of 6″-8″ should be sufficient. The width of the rigid backplate should be enough to prevent excessive lateral movement of the patient's lumbar spine. Generally, this means that the rigid backplate should be at least 9″ wide; however, other widths are also possible, depending on patient needs.

In the preferred embodiment of the invention, belt 110 is anywhere between 6″ and 8″ wide, and may be made of a heat-retention fabric to provide therapeutic heat to the injured area. The heat-retention fabric may be the far infrared TheraHeat fabric, to provide the appropriate amount of therapeutic heat. Alternately, any other heat retention fabric may be used. The belt is fastened around the patient's waist by means of primary fastener 130. This primary fastener 130 is preferably a Velcro fastener to allow easy attachment and removal without too much bulk. Other types of fasteners may also be used, as long as the fastener is sturdy enough to support the load without unfastening.

The pocket on the belt may be used for the rigid backplate, or may be used for a hot/cold gel pack to provide therapeutic heat or cold to the painful area. In an embodiment, a hot/cold gel pack of the appropriate size approximately equal to the size of the pocket is provided.

Supplementary fasteners 140 are located at the patient's sides in the preferred embodiment of the present invention. These fasteners allow the patient to tighten or loosen the orthosis without disturbing the primary fastener, Such fasteners may, as shown in the Figure, be straps that are attached to the belt on one end and that have a Velcro fastener on the other end that can be removably attached to the belt at several possible points along its length. Other fastening means, such as snaps or hooks, may also be used to attach the other end of the supplementary fastener 140 to the belt 110. In an alternate embodiment, the supplementary fasteners 140 are laces that are laced through holes in the belt and allow the patient to tighten the belt by pulling on the laces. There are preferably two supplementary fasteners, to allow for symmetrical adjustment; however, it is possible to use only one supplementary fastener, or more than two.

Primary fastener 130 includes a front pocket into which a semi-rigid front plate can be inserted. This provides extra support and compression for the patient's torso, as well as greater stability for patients with severe back problems.

The sides of the belt 110 may be made out of breathable elastic to provide greater comfort for the patient and to prevent sweating. The back of the belt is preferably made of polyester fabric. A heat-retention fabric such as TheraHeat may be used to provide therapeutic heat to the patient. A heat-reflection fabric, or a heated fabric, may also be used for that purpose.

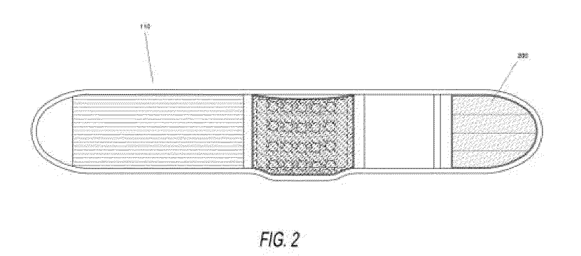

FIG. 2 shows a semi-transparent view of the lumbar orthosis lying flat. A semi-rigid front plate 200 is inserted into a pocket on belt 110. The semi-rigid front plate provides extra stability and compression; it is preferably made of a thin sheet of flexible plastic that is flexible enough to be comfortable for the patient while being rigid enough to provide some additional support for the patient's torso from the front. This is intended for patients with more severe back problems where just the rigid backplate is insufficient. The semi-rigid front plate is preferably made out of 1.5 mm thick LDPE, but may be made out of any other material that is flexible enough to conform to the contours of the patient's body, yet rigid enough to provide some additional support.

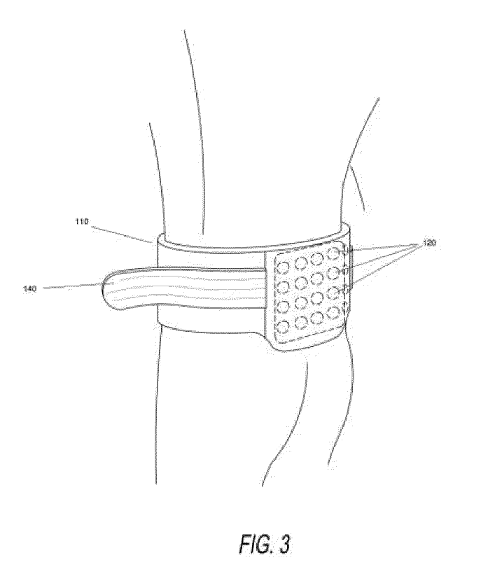

FIG. 3 shows the lumbar orthosis being worn by a patient. Permanent magnets 120 are attached to the belt 110 in the patient's lumbar area. The magnets provide a permanent magnetic field that improves circulation and assists in healing. The magnets may be placed in any pattern, and any quantity or shape of magnets may be used, as long as the magnetic field they provide is sufficient for a therapeutic effect. In the preferred embodiment, an array of disc-shaped magnets are attached to the belt in the patient's lumbar area, and placed on top of the rigid backplate so as to not interfere with the fit of the backplate. In alternate embodiments, the magnets may also be attached to the rigid backplate or embedded within the rigid backplate, or embedded within the pocket material. In other embodiments, the backplate itself may be magnetic.

In the preferred embodiment, an array of disc-shaped magnets is used. The magnets are preferably discs that are 20×3.5 mm in size and provide 500±100 Gauss per magnet. In the preferred embodiment, the material of the magnets is Y15 (approximately 85% Fe₂O₃ and 15% BaCO₃). However, any other magnets may be used as long as they provide a similar magnetic force, or a stronger magnetic force.

In another embodiment of the invention, permanent magnets are located in both the lumbar area and the abdominal area. The preferred number of magnets for that embodiment is 25 permanent magnets in the lumbar area and 6 permanent magnets in the abdominal area, each arranged in a rectangular array. This creates a three-dimensional magnetic field that has a greater therapeutic value for the patient. The array of magnets in the lumbar area preferably extends to the coccyx or close to the coccyx, but may be placed anywhere, In an embodiment, the location of the magnets is chosen by a clinician to configure the magnetic field in a way that is most therapeutic for the patient.

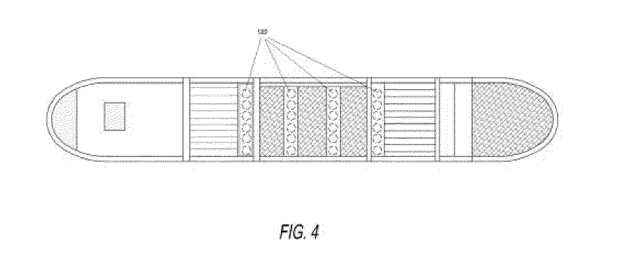

FIG. 4 shows a flat view of an alternate embodiment of the lumbar orthosis. In this embodiment, permanent magnets 120 are arranged in vertical lines rather than a single rectangular array. This provides greater comfort to the patient by making the orthosis more flexible without sacrificing the beneficial advantages of the magnetic field.

1. A magnetic lumbar orthosis, comprising:
a belt, said belt at least 6 inches wide, said belt comprising a first end and a second end;
a first fastener located at the first end;
a second fastener located at the second end, said second fastener capable of securely connecting to the first fastener;
a pocket attached to the belt approximately at the halfway point between the first end and the second end;
a rigid backplate that conforms to the shape of a patient's back, said rigid backplate removably inserted in the pocket;
at least one permanent magnet, said permanent magnet located in the same area as the rigid backplate.

2. The magnetic lumbar orthosis of claim 1, wherein the at least one permanent magnet emits a magnetic field of at least 400 Gauss.

3. The magnetic lumbar orthosis of claim 1, wherein the at least one permanent magnet is nonremovably attached to the pocket.

4. The magnetic lumbar orthosis of claim 1, further comprising:
a gel hot/cold pack approximately the same size as the pocket,
wherein the rigid backplate may be removed from the pocket and the gel hot/cold pack may be inserted into the pocket.

5. The magnetic lumbar orthosis of claim 3, wherein the rigid backplate has a first side and a second side, the first side facing a patient's body when in use,
wherein the at least one permanent magnet is nonremovably attached to the second side of the rigid backplate.

6. The magnetic lumbar orthosis of claim 3, wherein the at least one permanent magnet is embedded within the material of the rigid backplate.

7. The magnetic lumbar orthosis of claim 1, wherein the at least one permanent magnet is made of the Y15 alloy.

8. The magnetic lumbar orthosis of claim 1, comprising a plurality of permanent magnets arranged in an array.

9. The magnetic lumbar orthosis of claim 1, wherein the belt further comprises a front pocket located approximately in the area opposite the pocket, further comprising:
a front plate, said front plate inserted into the front pocket, said front plate being semi-rigid.

10. The magnetic lumbar orthosis of claim 9, wherein the front plate is made of LDPE.

11. The magnetic lumbar orthosis of claim 9, further comprising:
at least one permanent magnet attached to the front pocket.

12. The magnetic lumbar orthosis of claim 9, further comprising:
at least one permanent magnet attached to the front plate.

13. The magnetic lumbar orthosis of claim 12, wherein the front plate comprises a first side and a second side, the first side facing a patient's body when in use, wherein the at least one permanent magnet is attached to the second side.

14. The magnetic lumbar orthosis of claim 12, wherein the front plate comprises a first side and a second side, the first side facing a patient's body when in use, wherein the at least one permanent magnet is attached to the first side.

15. The magnetic lumbar orthosis of claim 12, wherein the at least one permanent magnet is embedded within the material of the front plate.

16. The magnetic lumbar orthosis of claim 1, wherein the belt is made of TheraHeat fabric.

17. The magnetic lumbar orthosis of claim 1, further comprising:
at least one supplementary fastener located on the belt at a location distinct from either the first fastener or the second fastener, said at least one supplementary fastener capable of tightening or loosening the belt without disturbing the first fastener or the second fastener.

18. The magnetic lumbar orthosis of claim 17, wherein each supplementary fastener comprises:
a first patch of a hook-and-loop fastener attached to the belt;
a strap attached to the belt on one end, said strap comprising a free end;
a second patch of a hook-and-loop fastener attached to the strap close to the free end, so that the first patch may be attached to the second patch.

19. The magnetic lumbar orthosis of claim 17, wherein each supplementary fastener comprises:
a first flap attached on one end to the belt at a first location distinct from the first fastener or the second fastener, said first location not being on the pocket, said first flap comprising at least one eyelet hole through which a lace may be threaded;
a second flap attached on one end to the belt at a second location distinct from the first fastener or the second fastener, said second location not being on the pocket, said second flap comprising at least one eyelet hole through which a lace may be threaded;
a lace threaded through at least one eyelet hole on the first flap and at least one eyelet hole on the second flap.

20. The magnetic lumbar orthosis of claim 17, comprising a first supplementary fastener at a location approximately one quarter of the length of the belt and a second supplementary fastener at a location approximately three quarters of the length of the belt.
